(19) United States
(12) Patent Application Publication
(10) Pub. No.: US 2025/0195588 A1
(43) Pub. Date: Jun. 19, 2025

(54) Ingestible Nutritive Compositions With Prebiotics, Probiotics, and Postbiotics

(71) Applicant: First Day Life, Inc., Austin, TX (US)

(72) Inventors: Bridgette Park, Granada Hills, CA (US); Shuyan Li, Austin, TX (US)

(21) Appl. No.: 18/544,239

(22) Filed: Dec. 18, 2023

(51) Int. Cl. A61K 35/747; A61K 9/00; A61K 35/00; A61K 35/742; A61K 36/185; A61P 1/14; A61P 31/04; A61P 31/16; A61P 37/04

(52) U.S. Cl. CPC A61K 35/747; A61K 9/0053; A61K 35/742; A61K 36/185; A61P 1/14; A61P 31/04; A61P 31/16; A61P 37/04; A61K 2035/115

(57) ABSTRACT

Compositions including prebiotics, probiotics, and postbiotics for oral administration and methods of using such compositions for improving health of subjects are provided.

### BACKGROUND OF THE INVENTION

The present invention generally relates to nutritional compositions comprising prebiotics, probiotics, and postbiotics for oral administration and methods for using the oral compositions in improving health of subjects (e.g., human subjects).

### BRIEF SUMMARY OF THE INVENTION

In an aspect of the invention, an orally ingestible nutritional composition for improving health is provided. The composition comprises an amount of gold kiwifruit powder, an amount of Bacillus coagulans, and an amount of heat-treated Lactobacillus paracasei. The gold kiwifruit powder within the composition may include or consist essentially of Livaux®. The Bacillus coagulans within the composition may include or consist essentially of SNZ 1969®. The heat-treated Lactobacillus paracasei within the composition may include or consist essentially of MCC1849.

In some embodiments, components of the inventive composition may be combined at a w/w ratio of between about 15:1 and about 35:1 gold kiwifruit powder: Bacillus coagulans. In some embodiments, components of the inventive composition may be combined at a w/w ratio of between about 5:1 and about 20:1 gold kiwifruit powder: Lactobacillus paracasei.

In some embodiments, components of the inventive composition may be combined at a w/w ratio of between about 1.5:1 and about 3:1 Lactobacillus paracasei:Bacillus coagulans.

In some embodiments, the orally ingestible compositions may take the form of gummies, tablets, capsules, liquids, suspensions, chewables, soft gels, sachets, powders, syrups, liquid suspensions, emulsions, or other solutions. In particular embodiments, the orally ingestible composition is in the form of an individual dosage form. The individual dosage form may take the form of a gummy, a tablet, a capsule, a soft gel, a sachet, or a chew. In particular embodiments, the individual dosage form is a gummy. In further embodiments, an individual oral dosage form may comprise between about 75 mg and 700 mg gold kiwifruit powder; between about 5 mg and 80 mg heat-treated Lactobacillus paracasei; and between about 1 mg and 40 mg Bacillus coagulans. In particular embodiments, the individual oral dosage form comprises about 125 mg gold kiwifruit powder; about 10 mg heat-treated Lactobacillus paracasei; and about 5 mg Bacillus coagulans. In particular embodiments, an individual oral dosage form may comprise an amount of gold kiwifruit powder, an amount of heat-treated Lactobacillus paracasei, and between about 100 million CFUs and 4 billion CFUs Bacillus coagulans. In particular embodiments, the individual oral dosage form comprises an amount of gold kiwifruit powder, an amount of heat-treated Lactobacillus paracasei, and about 500 million CFUs Bacillus coagulans.

In an aspect, there is a method for administering orally ingestible compositions to a subject in accordance with the disclosure. In some embodiments, the subject is a mammal. In some embodiments, the subject is a human. In some embodiments, the subject is an adult human. In particular embodiments, the subject is an adult human female. In particular embodiments, the subject is an adult human male. In particular embodiments, the subject is an elderly adult human. In still further embodiments, the subject is an elderly adult female. In still further embodiments, the subject is an elderly adult male. In some embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17.

Compositions according to embodiments herein may be administered a single time, daily on a continual basis, or multiple times per week depending on subject needs, subject body size, and on safety. Dosing may occur once, or only a weekly or monthly basis. Dosage level and frequency of administration protocols, compositions, as well as potential use of additional therapeutic or nutritive agents in combination can be adjusted during the course of administration. In an embodiment, compositions in accordance with the present inventions can be administered daily to a subject. In a particular embodiment, compositions in accordance with the present inventions can be administered multiple times per day to a subject.

In some embodiments, the subject receives, via oral administration of compositions in accordance with the disclosure herein, between about 75 mg and 2800 mg of gold kiwifruit powder per day.

In some embodiments, the subject receives, via oral administration of compositions in accordance with the disclosure herein, between about 5 mg and 320 mg of heat-treated Lactobacillus paracasei per day.

In some embodiments, the subject receives, via oral administration of compositions in accordance with the disclosure herein, between about 5 mg and 160 mg of Bacillus coagulans per day. In some embodiments, the subject receives between about 500 million CFUs and 16 billion CFUs Bacillus coagulans.

In an embodiment, a method of increasing the amount of Faecalibacterium prausnitzii within a digestive tract of a subject is provided. The method comprises orally administering, to the subject, an orally ingestible composition in accordance with the disclosure herein. In particular embodiments, the subject is a human. In particular embodiments, the subject is a human adult. In further embodiments, the subject is a human child under the age of 18. In some embodiments, the method comprises orally delivering to the subject an effective amount or concentration of one or more of gold kiwifruit powder, heat-treated Lactobacillus paracasei, and Bacillus coagulans to achieve an increase in the amount of Faecalibacterium prausnitzii within the digestive tract of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In an embodiment, a method of reducing constipation or relieving symptoms of constipation in a subject is provided. The method comprises orally administering, to the subject, an orally ingestible composition in accordance with the disclosure herein. In particular embodiments, the subject is a human. In particular embodiments, the subject is a human adult. In further embodiments, the subject is an adult human female. In particular embodiments, the subject is a human child under the age of 18.

In some embodiments, the method comprises orally delivering to the subject an effective amount or concentration of one or more of gold kiwifruit powder, heat-treated Lactobacillus paracasei, and Bacillus coagulans to achieve a reduction in constipation or relief of symptoms of constipation in the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments, the subject's frequency of complete spontaneous bowel movement is increased post-administration of the orally ingestible composition.

In some embodiments, the subject's stools are softened and/or improved in consistency post-administration of the orally ingestible composition as measured by the Bristol stool form scale.

In some embodiments, the subject's pain during defecation is reduced post-administration of the orally ingestible composition as measured by the constipation scoring system (CSS) scale.

In some embodiments, the subject's abdominal pain associated with constipation is reduced post-administration of the orally ingestible composition as measured by the constipation scoring system (CSS) scale.

In an embodiment, a method of reducing bacterial vaginosis symptoms in a human female subject is provided. The method comprises orally administering, to the human female subject, an orally ingestible composition in accordance with the disclosure herein. In particular embodiments, the subject is an adult human female. In particular embodiments, the subject is a female human child under the age of 18. In some embodiments, the method comprises orally delivering to the subject an effective amount or concentration of one or more of gold kiwifruit powder, heat-treated Lactobacillus paracasei, and Bacillus coagulans to reduce bacterial vaginosis symptoms in the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and billion CFUs Bacillus coagulans per day. In some embodiments, the human female subject is undergoing antibiotic treatment for bacterial vaginosis.

In an embodiment, a method of improving the immune response of a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In some embodiments, the method comprises orally delivering to the subject an effective amount or concentration of one or more of gold kiwifruit powder, Lactobacillus paracasei, and Bacillus coagulans to improve the immune response of the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments, the serum IL-12 is increased in the human subject post-administration of the orally ingestible composition.

In some embodiments, the production of IgA antibodies is increased in the human subject post-administration of the orally ingestible composition.

In some embodiments, incidence frequency of gastrointestinal (GI) infections and/or symptoms associated therewith is decreased in the human subject post-administration of the orally ingestible composition.

In some embodiments, incidence frequency of upper respiratory tract infections is decreased in the human subject post-administration of the orally ingestible composition.

In some embodiments, the incidence frequency of colds and cold-like illness in the human subject is decreased post-administration of the orally ingestible composition.

In some embodiments, the incidence frequency of influenza and influenza-like illness in the human subject is decreased post-administration of the orally ingestible composition.

In an embodiment, a method of improving oral health and reducing tooth decay in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In further embodiments, the human subject is an adult human male. In some embodiments, the method comprises orally delivering to the subject an effective amount or concentration of one or more of gold kiwifruit powder, heat-treated Lactobacillus paracasei, and Bacillus coagulans to improve the oral health of and reduce tooth decay in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments, salivary Streptococcus mutans content is decreased in the oral cavity of the human subject post-administration of the orally ingestible composition in accordance with the disclosure herein. In some embodiments, the incidence of tooth cavities within the oral cavity of the human subject is reduced following oral administration of the orally ingestible composition.

In some embodiments, the amount of plaque within the oral cavity of the human subject is reduced following oral administration of the orally ingestible composition.

In some embodiments, the gingival score of the oral cavity of the human subject is reduced following oral administration of the orally ingestible composition.

In an embodiment, a method of reducing gastrointestinal discomfort in a human subject is provided. The method comprises orally administering, to the subject, an orally ingestible composition in accordance with the disclosure herein. In particular embodiments, the subject is child under the age of 18. In particular embodiments, the subject is a human adult. In further embodiments, the subject is an adult human female. In further embodiments, the human subject is an adult human male. In some embodiments, the method comprises orally delivering to the subject an effective amount or concentration of one or more of gold kiwifruit powder, heat-treated Lactobacillus paracasei, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments, the human subject's total score on the Severity of Dyspepsia Assessment (SODA) scale is reduced following oral administration of the orally ingestible composition.

In some embodiments, the human subject's pain intensity subscore on the SODA scale is reduced following oral administration of the orally ingestible composition.

In some embodiments, the human subject's nonpain symptoms subscore on the SODA scale is reduced following oral administration of the orally ingestible composition.

### BRIEF DESCRIPTION OF THE SEVERAL VIEWS OF THE DRAWINGS

The foregoing summary, as well as the following detailed description of embodiments of the compositions and methods disclosed herein, will be better understood when read in conjunction with the appended drawings of exemplary embodiments. It should be understood, however, that the invention is not limited to the precise arrangements and instrumentalities shown.

In the drawings:

FIG. 1 describes a randomized controlled human trial on the effect of kiwifruit capsules consumptions on gut microflora in functionally constipated individuals. FIG. 1A lists a description of tested compositions and dosage amounts. FIG. 1B lists partial results of the study, underscoring the significant increase in abundance of Faecalibacterium prausnitzii in functionally constipated individuals.

FIG. 2 summarizes a clinical study on the effect of L. paracasei MCC1849 supplementation on symptoms of the common cold and mood states in healthy individuals. FIG. 2A displays the incidence, total number of days of symptoms, and severity of the common cold during the 12-week intervention in the protocol set. FIG. 2B depicts the incidence, total number of days of symptoms, and severity of the common cold during the 12-week intervention in the per protocol set sub-group (common cold once of more in the previous year).

FIG. 3 summarizes a clinically study on the safety and efficacy of B. coagulans SNZ 1969 supplementation in reducing infections and symptoms of illness in malnourished children.

FIG. 4 describes a clinical study of B. coagulans SNZ-1969 in the treatment of recurrent bacterial vaginosis (BV). Comparative analysis of the effect of treatment on symptomatic relief (FIG. 4A) and on Amsels criteria changes (FIG. 4B) are depicted for both groups.

FIG. 5 summarizes the effect of B. coagulans (L. sporogenes) on infantile (FIG. 5A) and neonatal (FIG. 5B) diarrhea.

FIG. 6 summarizes the effect of B. coagulans on oral health. Comparison of chlorexhidine, tulsi, and probiotic mouthwash on plaque (FIG. 6A) and gingival (FIG. 6B) status; FIG. 6C: Effect of B. coagulans, with or without vitamin B supplementation, on recurrent aphthous ulcerations; FIG. 6D: effect of B. coagulans on salivary mutans streptococci compared to control group (left panel) and between groups (right panel). FIG. 6E: Effect of B. coagulans on gingival index (left panel) and plaque index (right panel).

FIG. 7 Illustrates the effect of B. coagulans on gastrointestinal discomfort. FIG. 7A: Change from baseline to 30 days and day 37 in SODA and GSRS symptoms scores; FIG. 7B: Frequency of participants experiencing bloating symptom (left panel) or burping symptoms (right panel) in SODA scale at day 30; FIG. 7C: Frequency of participants experiencing heart burn symptom (left panel) and gas (right panel) in SODA scale at day 30.

### DETAILED DESCRIPTION OF THE INVENTION

Oral supplements and foods including probiotics, beneficial live microorganisms that confer health benefits on their “host” organisms (e.g., humans), have gained popularity as nutritional and health aids. However, to confer a substantial and/or lasting health benefit on those using them, the probiotics must be administered in an adequate amount and have an environment in which they can persist within the subject. It is often difficult to provide an adequate “stabilized” microbiome environment within a “host” organism to reap the full benefits of probiotic supplementation or consumption.

The interactions between probiotics, prebiotics, and postbiotics have recently garnered attention for the potential to address the issues associated with reaping the benefits of probiotics. Prebiotics may be considered to be probiotic “food”; that is, they are substrates selectively used by probiotic microorganisms in their life cycle maintenance, helping the probiotics survive during digestion to be delivered to the appropriate area of the digestive system. Prebiotics may also be used by the probiotic microorganisms to confer health benefits to the “host” organism. Most, if not, all prebiotic supplements utilize prebiotic fibers like fructooligosaccharides (FOS), xylooligosaccharides (XOS), galacto-oligosaccharides (GOS), or inulin due to low dosage requirements. However, these prebiotics are not considered FODMAP-friendly, as they are fermentable oligosaccharides which can cause unwanted GI side effects (e.g., bloating, gas).

By contrast, postbiotics may comprise inanimate or dead microorganisms, their respective parts, or waste byproducts produced by probiotic microorganisms that create a healthy microenvironment for probiotic or beneficial microorganisms to flourish. Postbiotics in the form of dead microorganisms may also confer similar health benefits to that of probiotics, but are not at risk of biological instability, since they are already non-viable. It is believed that the interplay between prebiotics, probiotics, and postbiotics may synergistically enhance the beneficial function each is known to confer separately, as the addition of pre- and postbiotics to probiotics is believed to stabilize the life cycle processes of the probiotic microorganisms.

Here, the inventors have combined pre-, pro-, and postbiotics in surprisingly efficacious manners, yielding beneficial nutritive compositions that demonstrate an enhanced, synergistic effect when consumed by a subject, leading to increased health benefits for the subject. The inventors have also developed surprisingly effective methods of using these beneficial compositions for increasing the health of subjects taking these compositions orally. Moreover, the inventors have used FODMAP-friendly prebiotics in compositions according to the disclosure herein to avoid the issues associated with typical prebiotic-containing foods and supplements.

### I. COMPOSITIONS

In an aspect of the invention, an orally ingestible nutritional composition for improving health is provided. The composition comprises an amount of gold kiwifruit powder (e.g., a prebiotic), an amount of Bacillus coagulans (a probiotic), and an amount of heat-treated Lactobacillus paracasei (postbiotic). The gold kiwifruit powder within the composition may include or consist essentially of Livaux®. The Bacillus coagulans within the composition may include or consist essentially of SNZ 1969®. The heat-treated Lactobacillus paracasei within the composition may include or consist essentially of MCC1849.

In further embodiments, an individual oral dosage form may comprise between about 75 mg and 700 mg gold kiwifruit powder; between about 5 mg and 80 mg heat-treated Lactobacillus paracasei; and between about 1 mg and 40 mg Bacillus coagulans. In further embodiments, an individual oral dosage form may comprise between about 75 mg and 100 mg gold kiwifruit powder; between about 5 mg and 15 mg heat-treated Lactobacillus paracasei; and between about 1 mg and 7.5 mg Bacillus coagulans. In further embodiments, an individual oral dosage form may comprise between about 100 mg and 200 mg gold kiwifruit powder; between about 15 mg and 25 mg heat-treated Lactobacillus paracasei; and between about 7.5 mg and 12.5 mg Bacillus coagulans. In further embodiments, an individual oral dosage form may comprise between about 200 mg and 300 mg gold kiwifruit powder; between about 25 mg and 45 mg heat-treated Lactobacillus paracasei; and between about 12.5 mg and 22.5 mg Bacillus coagulans. In further embodiments, an individual oral dosage form may comprise between about 300 mg and 500 mg gold kiwifruit powder; between about 45 mg and 60 mg heat-treated Lactobacillus paracasei; and between about 22.5 mg and 30 mg Bacillus coagulans. In further embodiments, an individual oral dosage form may comprise between about 500 mg and 700 mg gold kiwifruit powder; between about 60 mg and 80 mg heat-treated Lactobacillus paracasei; and between about 30 mg and 40 mg Bacillus coagulans.

In particular embodiments, the individual oral dosage form comprises about 75 mg gold kiwifruit powder; about 5 mg heat-treated Lactobacillus paracasei; and about 2.5 mg Bacillus coagulans. In particular embodiments, the individual oral dosage form comprises about 125 mg gold kiwifruit powder; about 10 mg heat-treated Lactobacillus paracasei; and about 5 mg Bacillus coagulans. In particular embodiments, the individual oral dosage form comprises about 250 mg gold kiwifruit powder; about 20 mg heat-treated Lactobacillus paracasei; and about 10 mg Bacillus coagulans. In particular embodiments, the individual oral dosage form comprises about 500 mg gold kiwifruit powder; about 40 mg heat-treated Lactobacillus paracasei; and about 20 mg Bacillus coagulans. In particular embodiments, the individual oral dosage form comprises about 700 mg gold kiwifruit powder; about 56 mg heat-treated Lactobacillus paracasei; and about 28 mg Bacillus coagulans.

According to certain embodiments of the inventive composition, the compositions are formulated such that they cause improvement in health markers within a subject consuming them (e.g., a demonstrable improvement in digestive health). Health improvements may be measured by improvement in symptoms of particular disorders (e.g., reduction in the amount of Streptococcus mutans within the oral cavity), or increase in markers of health (e.g., increase in the amount of Faecalibacterium prausnitzii within a digestive tract).

In a further embodiment, the composition includes concentrations or amounts of the pre-, pro-, and postbiotics that are therapeutically effective to cause improvement in health markers within the subject.

In a preferred embodiment of the composition, the composition includes gold kiwifruit powder as a prebiotic. Gold kiwifruit powder possesses well-demonstrated tolerability in vivo and is FODMAP-friendly. It is also shown to increase the presence of Faecalibacterium prausnitzii in the microbiome, a beneficial gut probiotic microorganism. Gold kiwifruit powder has also been shown to improve gut transit, contribute to fecal bulking, and increase mucus production in the gut. Clinically or therapeutically effective amounts, or amounts sufficient to cause improvement in health markers within the subject, of the gold kiwifruit powder in compositions according to the invention herein include from about 75 mg to about 2800 mg of gold kiwifruit powder per daily total dose, from about 75 mg to about 2500 mg per daily total dose; from about 75 mg to about 2250 mg per daily total dose; from about 75 mg to about 2000 mg per daily total dose; from about 75 mg to about 1750 mg per daily total dose; from about 75 mg to about 1500 mg per daily total dose; from about 75 mg to about 1250 mg per daily total dose; from about 75 mg to about 1000 mg per daily total dose; from about 75 mg to about 750 mg per daily total dose; from about 75 mg to about 500 mg per daily dose; or from about 75 mg to about 250 mg per daily total dose. The gold kiwifruit powder may comprise Livaux®.

In particular embodiments, clinically or therapeutically effective amounts, or amounts sufficient to cause improvement in health markers within the subject, of the gold kiwifruit powder in compositions according to the invention herein include about 2800 mg of gold kiwifruit powder per daily total dose, about 2500 mg per daily total dose; about 2250 mg per daily total dose; about 2000 mg per daily total dose; about 1750 mg per daily total dose; about 1500 mg per daily total dose; about 1250 mg per daily total dose; about 1000 mg per daily total dose; about 750 mg per daily total dose; about 500 mg per daily dose; about 450 mg per daily total dose; about 400 mg per daily total dose; about 350 mg per daily total dose; about 300 mg per daily total dose; about 250 mg per daily total dose; about 225 mg per daily total dose; about 200 mg per daily total dose; about 175 mg per daily total dose; about 150 mg per daily total dose; about 125 mg per daily total dose; about 100 mg per daily total dose; or about 75 mg per daily total dose.

In a preferred embodiment of the composition, the composition includes Bacillus coagulans as a probiotic. In particularly preferred embodiments, the B. coagulans comprises SNZ 1969®. SNZ 1969® has been shown to improve gut, oral, and immune health across multiple therapeutic endpoints and in different human populations, including in children.

Clinically or therapeutically effective amounts, or amounts sufficient to cause improvement in health markers within the subject, of the Bacillus coagulans in compositions according to the invention herein include from about 5 mg to about 160 mg of Bacillus coagulans per daily total dose, from about 5 mg to about 150 mg per daily total dose; from about 5 mg to about 140 mg per daily total dose; from about 5 mg to about 130 mg per daily total dose; from about 5 mg to about 120 mg per daily total dose; from about 5 mg to about 110 mg per daily total dose; from about 5 mg to about 100 mg per daily total dose; from about 5 mg to about 90 mg per daily total dose; from about 5 mg to about 80 mg per daily total dose; from about 5 mg to about 70 mg per daily total dose; from about 5 mg to about 60 mg per daily total dose; from about 5 mg to about 50 mg per daily total dose; from about 5 mg to about 40 mg per daily total dose; from about 5 mg to about 30 mg per daily total dose; or from about 5 mg to about 20 mg per daily total dose. Clinically or therapeutically effective amounts, or amounts sufficient to cause improvement in health markers within the subject, of the Bacillus coagulans in compositions according to the invention herein include between about 500 million CFUs and 16 billion CFUs Bacillus coagulans per daily total dose, from about 500 million CFUs to about 15 billion CFUs Bacillus coagulans per daily total dose, from about 500 million CFUs to about 14 billion CFUs Bacillus coagulans per daily total dose, from about 500 million CFUs to about 13 billion CFUs Bacillus coagulans per daily total dose, from about 500 million CFUs to about 12 billion CFUs Bacillus coagulans per daily total dose, from about 500 million CFUs to about 11 billion CFUs Bacillus coagulans per daily total dose, from about 500 million CFUs to about 10 billion CFUs Bacillus coagulans per daily total dose, from about 500 million CFUs to about 9 billion CFUs Bacillus coagulans per daily total dose, from about 500 million CFUs to about 8 billion CFUs Bacillus coagulans per daily total dose, from about 500 million CFUs to about 7 billion CFUs Bacillus coagulans per daily total dose, from about 500 million CFUs to about 6 billion CFUs Bacillus coagulans per daily total dose, from about 500 million CFUs to about 5 billion CFUs Bacillus coagulans per daily total dose, from about 500 million CFUs to about 4 billion CFUs Bacillus coagulans per daily total dose, from about 500 million CFUs to about 3 billion CFUs Bacillus coagulans per daily total dose, from about 500 million CFUs to about 2 billion CFUs Bacillus coagulans per daily total dose, from about 750 million CFUs to about 10 billion CFUs Bacillus coagulans per daily total dose, from about 750 million CFUs to about 5 billion CFUs Bacillus coagulans per daily total dose, from about 750 million CFUs to about 2 billion CFUs Bacillus coagulans per daily total dose, from about 1 billion CFUs to about 15 billion CFUs Bacillus coagulans per daily total dose, from about 1 billion CFUs to about 10 billion CFUs Bacillus coagulans per daily total dose, from about 1 billion CFUs to about 5 billion CFUs Bacillus coagulans per daily total dose, about 500 million CFUs, about 750 million CFUS, about 1 billion CFUs, about 1.25 billion CFUs, about 1.5 billion CFUS, about 1.75 billion CFUs, about 2 billion CFUs, about 2.25 billion CFUs, about 2.5 billion CFUS, about 2.75 billion CFUs, about 3 billion CFUs, about 3.25 billion CFUs, about 3.5 billion CFUS, about 3.75 billion CFUs, about 4 billion CFUs, about 4.25 billion CFUs, about 4.5 billion CFUS, about 4.75 billion CFUs, about 5 billion CFUs, about 5.25 billion CFUs, about 5.5 billion CFUS, about 5.75 billion CFUs, about 6 billion CFUs, about 7 billion CFUs, about 8 billion CFUs, about 9 billion CFUs, about 10 billion CFUs, about 11 billion CFUs, about 12 billion CFUs, about 13 billion CFUs, about 14 billion CFUs, about 15 billion CFUs, or about 16 billion CFUs. The B. coagulans may comprise SNZ 1969®.

In particular embodiments, clinically or therapeutically effective amounts, or amounts sufficient to cause improvement in health markers within the subject, of the Bacillus coagulans in compositions according to the invention herein include about 160 mg of Bacillus coagulans per daily total dose, about 150 mg per daily total dose; about 140 mg per daily total dose; about 130 mg per daily total dose; about 120 mg per daily total dose; about 110 mg per daily total dose; about 100 mg per daily total dose; about 90 mg per daily total dose; about 80 mg per daily total dose; about 70 mg per daily total dose; about 60 mg per daily total dose; about 50 mg per daily total dose; about 40 mg per daily total dose; about 30 mg per daily total dose; about 20 mg per daily total dose; about 15 mg per daily total dose; about 10 mg per daily total dose; or about 5 mg per daily total dose. The B. coagulans may comprise SNZ 1969®.

In a preferred embodiment of the composition, the composition includes heat-treated (i.e., killed) Lactobacillus paracasei as a postbiotic. In particularly preferred embodiments, the heat-treated L. paracasei comprises MCC1849. MCC1849 has been shown to support immunomodulatory effects across multiple therapeutic endpoints and in different human populations.

Clinically or therapeutically effective amounts, or amounts sufficient to cause beneficial immunomodulatory effects within the subject, of the L. paracasei in compositions according to the invention herein include from about 5 mg to about 320 mg of L. paracasei per daily total dose, from about 5 mg to about 300 mg per daily total dose; from about 5 mg to about 280 mg per daily total dose; from about 5 mg to about 260 mg per daily total dose; from about 5 mg to about 240 mg per daily total dose; from about 5 mg to about 220 mg per daily total dose; from about 5 mg to about 200 mg per daily total dose; from about 5 mg to about 180 mg per daily total dose; from about 5 mg to about 160 mg per daily total dose; from about 5 mg to about 140 mg per daily total dose; from about 5 mg to about 120 mg per daily total dose; from about 5 mg to about 100 mg per daily total dose; from about 5 mg to about 80 mg per daily total dose; from about 5 mg to about 60 mg per daily total dose; from about 5 mg to about 40 mg per daily total dose; or from about 5 mg to about 20 mg per daily total dose.

In particular embodiments, clinically or therapeutically effective amounts, or amounts sufficient to cause beneficial immunomodulatory effects within the subject, of the L. paracasei in compositions according to the invention herein include about 320 mg of L. paracasei per daily total dose, about 300 mg per daily total dose; about 280 mg per daily total dose; about 260 mg per daily total dose; about 240 mg per daily total dose; about 220 mg per daily total dose; about 200 mg per daily total dose; about 180 mg per daily total dose; about 160 mg per daily total dose; about 140 mg per daily total dose; about 120 mg per daily total dose; about 100 mg per daily total dose; about 80 mg per daily total dose; about 60 mg per daily total dose about 40 mg per daily total dose; about 35 mg per daily total dose; about 30 mg per daily total dose; about 25 mg per daily total dose; about 20 mg per daily total dose; about 15 mg per daily total dose; about 10 mg per daily total dose; or about 5 mg per daily total dose. The L. paracasei may comprise MCC1849.

Compositions according to embodiments of the invention may include combinations of components each in clinically or therapeutically effective amounts. For example, in some compositional embodiments, each of the gold kiwifruit powder, the B. coagulans, and the L. paracasei may be included in therapeutically effective amounts in composition.

In some embodiments, components of the inventive composition may be combined at a w/w ratio of between about 15:1 and about 35:1 gold kiwifruit powder: Bacillus coagulans. In some embodiments, components of the inventive composition May be combined at a w/w ratio of between about 15:1 and about 30:1 gold kiwifruit powder: Bacillus coagulans. In some embodiments, components of the inventive composition May be combined at a w/w ratio of between about 15:1 and about 25:1 gold kiwifruit powder: Bacillus coagulans. In some embodiments, components of the inventive composition may be combined at a w/w ratio of between about 15:1 and about 20:1 gold kiwifruit powder: Bacillus coagulans. In some embodiments, components of the inventive composition may be combined at a w/w ratio of between about 18:1 and about 32:1 gold kiwifruit powder: Bacillus coagulans. In some embodiments, components of the inventive composition may be combined at a w/w ratio of between about 20:1 and about 30:1 gold kiwifruit powder: Bacillus coagulans. In some embodiments, components of the inventive composition May be combined at a w/w ratio of between about 22.5:1 and about 27.5:1 gold kiwifruit powder: Bacillus coagulans.

In some embodiments, components of the inventive composition may be combined at a w/w ratio of between about 5:1 and about 20:1 gold kiwifruit powder: Lactobacillus paracasei. In some embodiments, components of the inventive composition may be combined at a w/w ratio of between about 5:1 and about 17.5:1 gold kiwifruit powder: Lactobacillus paracasei. In some embodiments, components of the inventive composition may be combined at a w/w ratio of between about 5:1 and about 15.5:1 gold kiwifruit powder: Lactobacillus paracasei. In some embodiments, components of the inventive composition may be combined at a w/w ratio of between about 5:1 and about 12.5:1 gold kiwifruit powder: Lactobacillus paracasei. In some embodiments, components of the inventive composition may be combined at a w/w ratio of between about 5:1 and about 10:1 gold kiwifruit powder: Lactobacillus paracasei. In some embodiments, components of the inventive composition may be combined at a w/w ratio of between about 7:1 and about 15:1 gold kiwifruit powder: Lactobacillus paracasei. In some embodiments, components of the inventive composition may be combined at a w/w ratio of between about 10:1 and about 13:1 gold kiwifruit powder: Lactobacillus paracasei.

In some embodiments, components of the inventive composition may be combined at a w/w ratio of between about 1.5:1 and about 3:1 Lactobacillus paracasei:Bacillus coagulans. In some embodiments, components of the inventive composition May be combined at a w/w ratio of between about 1.5:1 and about 2.75:1 Lactobacillus paracasei:Bacillus coagulans. In some embodiments, components of the inventive composition may be combined at a w/w ratio of between about 1.5:1 and about 2.5:1 Lactobacillus paracasei:Bacillus coagulans. In some embodiments, components of the inventive composition may be combined at a w/w ratio of between about 1.5:1 and about 2.25:1 Lactobacillus paracasei:Bacillus coagulans. In some embodiments, components of the inventive composition may be combined at a w/w ratio of between about 1.5:1 and about 2:1 Lactobacillus paracasei:Bacillus coagulans. In some embodiments, components of the inventive composition May be combined at a w/w ratio of between about 1.5:1 and about 1.75:1 Lactobacillus paracasei:Bacillus coagulans. In some embodiments, components of the inventive composition may be combined at a w/w ratio of between about 1.7:1 and about 2.5:1 Lactobacillus paracasei: Bacillus coagulans. In some embodiments, components of the inventive composition may be combined at a w/w ratio of between about 1.9:1 and about 2.1:1 Lactobacillus paracasei:Bacillus coagulans.

Compositions according to certain embodiments herein may be formulated as solid or liquid dosage forms. Dosage forms may be formulated for oral delivery. Oral delivery methods for compositions according to certain embodiments of the invention herein may include gummies, tablets, capsules, liquids, suspensions, chewables, soft gels, sachets, powders, syrups, liquid suspensions, emulsions, or other solutions. In particular embodiments, the orally ingestible composition is in the form of an individual dosage form. The individual dosage form may take the form of a gummy, a tablet, a capsule, or a chew. In particular embodiments, the individual dosage form is a gummy.

### II. METHODS

In an embodiment, a method of increasing the amount of Faecalibacterium prausnitzii within a digestive tract of a subject is provided. The method comprises orally administering, to the subject, an orally ingestible composition in accordance with the disclosure herein. In particular embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female. In some embodiments of increasing the amount of Faecalibacterium prausnitzii within a digestive tract of a subject, the method comprises orally delivering to the subject an effective amount or concentration of one or more of gold kiwifruit powder, Lactobacillus paracasei, and Bacillus coagulans to achieve an increase in the amount of Faecalibacterium prausnitzii within the digestive tract of the subject. In some embodiments of the method of increasing the amount of Faecalibacterium prausnitzii within a digestive tract of a subject, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In an embodiment, a method of reducing constipation or relieving symptoms of constipation in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female.

In some embodiments, the method of reducing constipation or relieving symptoms of constipation in a subject comprises orally delivering to the subject an effective amount or concentration of one or more of gold kiwifruit powder, Lactobacillus paracasei, and Bacillus coagulans to achieve a reduction in constipation or relief of symptoms of constipation in the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments of the method of reducing constipation or relieving symptoms of constipation in a subject, the subject's frequency of defecation is increased post-administration of the orally ingestible composition. In some embodiments, the subject's frequency of defecation is increased post-administration to at least three bowel movements per week.

In some embodiments of the method of reducing constipation or relieving symptoms of constipation in a subject, the subject's frequency of complete spontaneous bowel movements is increased. In some embodiments, the subject's frequency of complete spontaneous bowel movements is increased post-administration to at least three complete spontaneous bowel movements per week.

In some embodiments of the method of reducing constipation or relieving symptoms of constipation in a subject, the subject's stools are softened and/or improved in consistency post-administration of the orally ingestible composition as measured by the Bristol stool form scale. In some embodiments, the subject's stool consistency post-administration achieves a 3 or 4 on the Bristol stool form scale.

In an embodiment, a method of improving immune health in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female.

In some embodiments, the method of improving immune health in a subject comprises orally delivering to the subject an effective amount or concentration of one or more of gold kiwifruit powder, Lactobacillus paracasei, and Bacillus coagulans to achieve an improvement in immune health of a subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments of the method of improving immune health in a subject, the incidence frequency of colds and/or cold symptoms in the subject are decreased post-administration of the orally ingestible composition. In some embodiments, the subject's frequency of experiencing a cold and/or exhibiting cold symptoms is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune health in a subject, the incidence frequency of influenza and/or influenza symptoms in the subject are decreased post-administration of the orally ingestible composition. In some embodiments, the subject's frequency of experiencing influenza and/or exhibiting influenza symptoms is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune health in a subject, the incidence frequency of upper respiratory infections and/or symptoms thereof in the subject are decreased post-administration of the orally ingestible composition. In some embodiments, the subject's frequency of experiencing upper respiratory infections and/or symptoms thereof is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune health in a subject, the incidence frequency of gastrointestinal (GI) infections and/or symptoms thereof in the subject are decreased post-administration of the orally ingestible composition. In some embodiments, the subject's frequency of experiencing GI infections and/or symptoms thereof is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune health in a subject, the expression of IL-12 in the subject is increased in the subject post-administration of the orally ingestible composition. In some embodiments, the subject's IL-12 expression levels are increased by at least 1%, by at least 3%, by at least 5%, by at least 10%, by at least 15%, by at least 20%, by at least 25%, by at least 30%, by at least 35%, by at least 40%, by at least 45%, or by at least 50% post-administration of the orally ingestible composition.

In some embodiments of the method of improving immune health in a subject, the production of IgA antibodies in the subject is increased in the subject post-administration of the orally ingestible composition. In some embodiments, the subject's production of IgA antibodies is increased by at least 1%, by at least 3%, by at least 5%, by at least 10%, by at least 15%, by at least 20%, by at least 25%, by at least 30%, by at least 35%, by at least 40%, by at least 45%, or by at least 50% post-administration of the orally ingestible composition.

In an embodiment, a method of improving oral health in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female.

In some embodiments, the method of improving oral health in a subject comprises orally delivering to the subject an effective amount or concentration of one or more of gold kiwifruit powder, Lactobacillus paracasei, and Bacillus coagulans to achieve an improvement in oral health of a subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments of the method of improving oral health in a subject, the amount of Streptococcus mutans within the oral cavity of the subject is reduced post-9 administration of the orally ingestible composition. In some embodiments, the amount of Streptococcus mutans within the oral cavity of the subject is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving oral health in a subject, the incidence frequency of tooth cavities within the oral cavity of the subject is decreased post-administration of the orally ingestible composition. In some embodiments, the subject's frequency of experiencing tooth cavities is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving oral health in a subject, the amount of plaque within the oral cavity of the subject is reduced post-administration of the orally ingestible composition. In some embodiments, the amount of plaque within the oral cavity of the subject is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving oral health in a subject, the gingival score of the oral cavity of the subject is decreased post-administration of the orally ingestible composition. In some embodiments, the subject's gingival score is reduced by at least 5%, at least 10%, at least 15%, at least 20%, at least 25%, at least 30%, at least 35%, at least 40%, at least 45%, or at least 50% post-administration of the orally ingestible composition.

In an embodiment, a method of reducing bacterial vaginosis symptoms in a human female subject is provided. The method comprises orally administering, to the human female subject, an orally ingestible composition in accordance with the disclosure herein. In particular embodiments, the subject is a human female child under the age of 18. In particular embodiments, the subject is a human female child between the ages of 0 and 3. In particular embodiments, the subject is a human female child between the ages of 0 and 5. In particular embodiments, the subject is a human female child between the ages of 1 and 5. In particular embodiments, the subject is a human female child between the ages of 6 and 10. In particular embodiments, the subject is a human female child between the ages of 11 and 15. In particular embodiments, the subject is a human female child between the ages of 15 and 17. In particular embodiments, the subject is an adult human female.

In some embodiments, the method of reducing bacterial vaginosis symptoms comprises orally delivering to the subject an effective amount or concentration of one or more of gold kiwifruit powder, Lactobacillus paracasei, and Bacillus coagulans to reduce bacterial vaginosis symptoms in the subject. In some embodiments, the method of reducing bacterial vaginosis symptoms in a human female subject comprises orally delivering to the subject an effective amount or concentration of one or more of gold kiwifruit powder, Lactobacillus paracasei, and Bacillus coagulans to achieve a reduction in the bacterial vaginosis symptoms of the human female subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day. In some embodiments, the human female subject is undergoing antibiotic treatment for bacterial vaginosis.

In an embodiment, a method of reducing gastrointestinal discomfort in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female.

In some embodiments, the method of reducing gastrointestinal discomfort in a subject comprises orally delivering to the subject an effective amount or concentration of one or more of gold kiwifruit powder, Lactobacillus paracasei, and Bacillus coagulans to achieve a reduction in gastrointestinal discomfort of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactobacillus paracasei per day. In some embodiments, the subject receives, via oral administration of compositions in accordance with the disclosure herein, between about 5 mg and 160 mg of Bacillus coagulans per day. In some embodiments, the subject receives, via oral administration of compositions in accordance with the disclosure herein, between about 500 million CFUs and 16 billion CFUs Bacillus coagulans per day.

In some embodiments of the method of reducing gastrointestinal discomfort in a subject, the subject's total score on the Severity of Dyspepsia Assessment (SODA) scale is reduced post-administration of the orally ingestible composition. In some embodiments, the subject's total score on the SODA scale is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of reducing gastrointestinal discomfort in a subject, the subject's nonpain symptoms subscore on the SODA scale is reduced post-administration of the orally ingestible composition. In some embodiments, the subject's nonpain symptoms subscore on the SODA scale is reduced by at least 5%, at least 10%, at least 15%, at least 20%, at least 25%, at least 30%, at least 35%, at least 40%, at least 45%, or at least 50% post-administration of the orally ingestible composition.

### III. EXAMPLES

### Example 1: Effect of Gold Kiwifruit Capsules Consumptions on Gut Microflora in Functionally Constipated Individuals

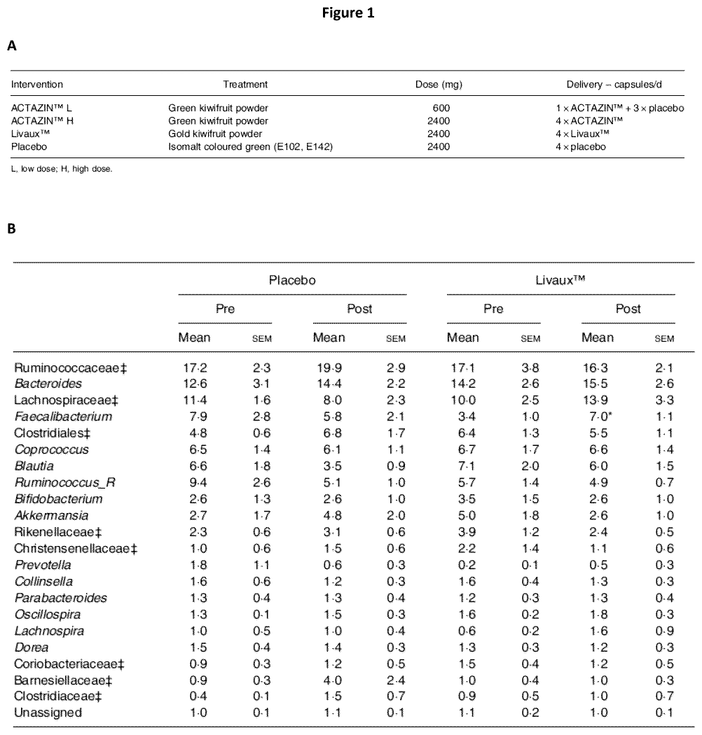

A randomized controlled human trial demonstrated that consumption of gold kiwifruit capsules (600 mg/capsule, up to 2400 mg/day for 14 days vs. isomalt placebo) significantly increased Faecalibacterium bacterial group abundance in functionally constipated subjects (see FIG. 1, from Blatchford et al., (2017) Consumption of kiwifruit capsules increases Faecalibacterium prausnitzii abundance in functionally constipated individuals: a randomised controlled human trial, Journal of Nutritional Science (2017), vol. 6, e52, page 1 of 10, herein incorporated by reference in its entirety). The study design was a randomised double-blind placebo-controlled cross-over trial with participants consuming four different interventions for 4 weeks each, with a 2-week washout between each intervention. The interventions were delivered in 4×600 mg capsules supplied by Anagenix Ltd prepared to look identical to preserve intervention blinding.

Briefly, participants consumed four different intervention combinations: placebo (isomalt coloured green) (2400 mg/d), ACTAZIN™ L (600 mg/d), ACTAZIN™ H (2400 mg/d) and Livaux™ (2400 mg/d) for 28 d each intervention, with a 14-day washout period between each treatment phase. ACTAZIN™ L (low dose, green kiwifruit) and ACTAZIN™ H (high dose, green kiwifruit) were formulated from cold-processed Actinidia chinensis var. deliciosa ‘Hayward’ green kiwifruit and Livaux™ was formulated from cold-processed Actinidia chinensis var. chinensis ‘Zesy002’ gold-fleshed kiwifruit. The placebo ingredient was isomalt (1-O-α-Dglucopyranosyl-D-mannitol). Subjects were asked to exclude high-fibre dietary supplements such as Metamucil, Benefibre and Phloe as well as maintaining their habitual dietary intakes and physical activity habits and to refrain from eating fresh kiwifruit for the study period. At the beginning and end of each 4-week intervention period, participants were asked to provide a faecal sample. The washout period of 2 weeks was chosen to allow sufficient time to return bowel habits to baseline for the parameters measured (microbial ecology, microbial metabolites and SmartPill® pH measurements).

This study investigated the impact of ACTAZIN™ green (2400 and 600 mg) and Livaux™ (2400 mg) gold kiwifruit supplements on faecal microbial composition and metabolites in healthy and functionally constipated (FC) participants. The participants were recruited into the healthy group (n 20; one of whom did not complete the study) and the FC group (n 9), each of whom consumed all the treatments and a placebo (isomalt) for 4 weeks in a randomised cross-over design interspersed with 2-week washout periods. Modification of faecal microbiota composition and metabolism was determined by 16S rRNA gene sequencing and GC, and colonic pH was calculated using SmartPill® wireless motility capsules. A total of thirty-two taxa were measured at greater than 1% abundance in at least one sample, ten of which differed significantly between the baseline healthy and FC groups. Specifically, Bacteroidales and Roseburia spp. were significantly more abundant (P<0.05) in the healthy group, and taxa including Ruminococcaceae, Dorea spp. and Akkermansia spp. were significantly more abundant (P<0.05) in the FC group. In the FC group, Faecalibacterium prausnitzii abundance significantly increased (P=0.024) from 3.4 to 7.0% following Livaux™ supplementation, with eight of the nine participants showing a net increase. Lower proportions of F. prausnitzii are often associated with gastrointestinal disorders. The discovery that Livaux™ supplementation increased F. prausnitzii abundance offers a potential strategy for improving gut microbiota composition, as F. prausnitzii is a butyrate producer and has also been shown to exert anti-inflammatory effects in many studies.

### Example 2: Effect of Lactobacillus paracasei on Common Cold Symptoms and Mood States in Individuals

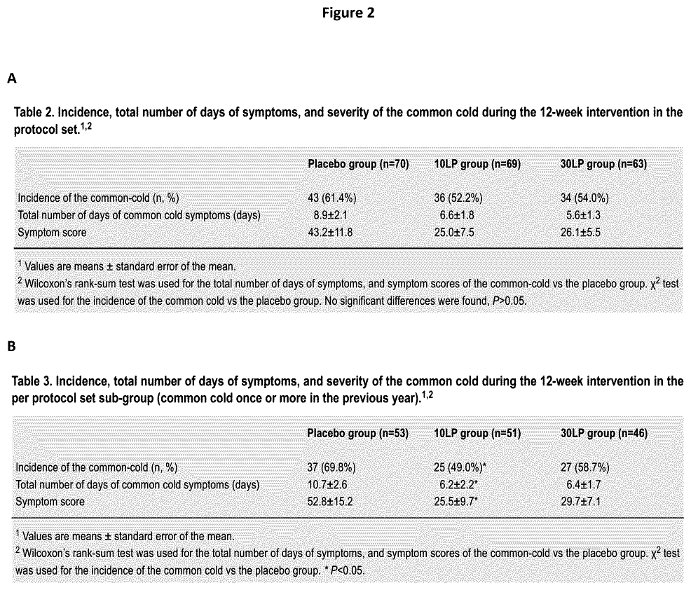

A total of 241 participants were randomized to receive 1×10¹⁰ heat-killed L. paracasei MCC1849 cell powder (10LP), 3×10¹⁰ heat-killed L. paracasei MCC1849 cell powder (30LP), or placebo powder without any L. paracasei cells once daily for 12 weeks based on the incidence of the common cold in the previous year, so that the risk of the incidence was equal among the groups. The incidence and severity of common cold symptoms were rated daily in a subject diary. Salivary secretory immunoglobulin A concentrations and saliva flow rates were analyzed at 0 and 6 weeks. The Profile of Mood States (POMS) was assessed using POMS 2 0, 6, and 12 weeks after the intervention. No significant differences were observed in the incidence of the common cold among the groups (FIG. 2A). In a prespecified subgroup of subjects who had the common cold in the previous year, the incidence, total number of days of symptoms, and symptom scores of the common cold significantly improved in the 10LP-intake group, and were slightly lower in the 30LP-intake group than in the placebo group (FIG. 2B). The level of deterioration in the positive mood state caused by stress was less in the MCC1849-intake group than in the placebo group. These results indicate that L. paracasei MCC1849 has the potential to improve resistance to common cold infections in susceptible subjects and maintain a desirable mood state, even under mental stress conditions. See Murata et al., Effects of paraprobiotic Lactobacillus paracasei MCC1849 supplementation on symptoms of the common cold and mood states in healthy adults, Beneficial Microbes, 2018; 9 (6): 855-864, herein incorporated by reference in its entirety.

### Example 3: Effect of Bacillus coagulans on Immunity of Moderately Malnourished Children

A total of 82 malnourished children aged 1-5 years with evidence of moderate malnutrition (having a weight-for-height z-score of −3.0 to less than −2.0), were enrolled in the study, which was randomized into Group A—probiotic Bacillus coagulans SNZ 1969 (n=41) and Group B—Placebo (n=41). Malnourished children participated in this study were randomized in a 1:1 ratio to the probiotic arm (Bacillus coagulans SNZ 1969) or placebo arm to receive 5 mL syrup containing 300 million CFU-thrice daily for a period of 3 months. Each 5 mL syrup containing 300 million CFU of Bacillus coagulans SNZ 1969 was given thrice daily for a period of 3 months. The study consisted of a baseline visit for screening and randomization followed by follow up visits I, II, & III on day 30, day 60, and day 90, respectively. Subject diaries were used to monitor the compliance of study medications.

The children experiencing vomiting and diarrhea were markedly lower in the probiotic group as compared to placebo, but statistical significance (p<0.05) was achieved only for “Children experiencing Diarrhea”. Other groups, like “Children experiencing AGI” in the 3rd month & “AGI lasting more than 2 days” also demonstrated a positive trend for probiotic as compared to placebo, but without statistical significance. These findings support the beneficial role of Bacillus coagulans SNZ 1969 in improvement of gut immunity. However, there is no difference in the children experiencing Respiratory infections in either group, which could be explained by their malnourishment/short treatment duration of 3 months and the long time taken for subject recruitment across 2-3 seasons in consecutive years.

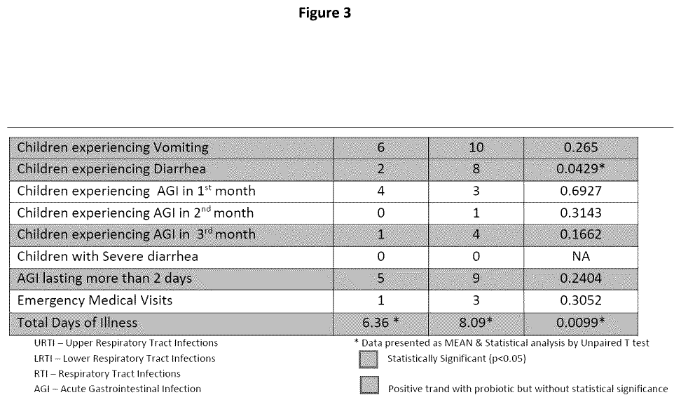

An unpaired t-test was performed on one study parameter, “Total days of Illness.” A statistically significant reduction was observed in the total number of days of illness in the group that was administered probiotic Bacillus coagulans SNZ 1969 when compared with the placebo group (p=. 009923) (FIG. 3). This reduction in the total days of illness in the probiotic group as compared to placebo may be attributed to the positive immune modulation of Bacillus coagulans SNZ 1969 bolstering the ability to resist a particular disease by improving immunity in malnourished children. See A randomized, double blind, placebo-controlled clinical study to evaluate the efficacy & safety of supplementation with Bacillus coagulans SNZ 1969 in reducing infections in malnourished children, herein incorporated by reference in its entirety.

### Example 4: Effect of Bacillus coagulans SNZ-1969 in the Treatment of Recurrent Bacterial Vaginosis

The present study aimed to evaluate the efficacy of Bacillus coagulans (SNZ-1969) against BV and the ability of this bacterial species to prevent BV recurrence in women aged 18-41 years. The study assessed 173 women, and of these women, 120 showed recurrent BV according to Nugent's and Amsel's criteria. These 120 women were randomly assigned to the following study arms: metronidazole arm (400 mg BID×7 days) and metronidazole (400 mg BID×7 days)+B. coagulans arm (120 million CFU/tablet×TID×21 days).

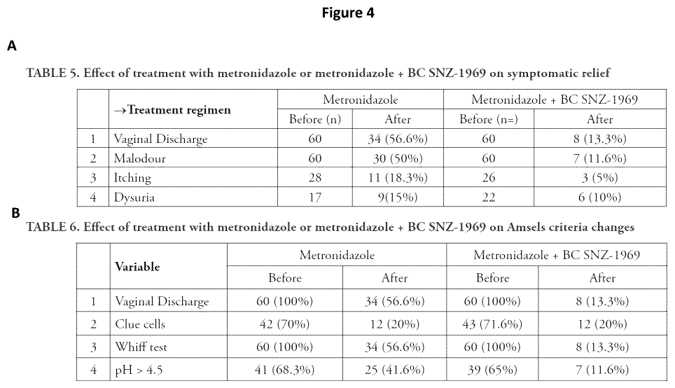

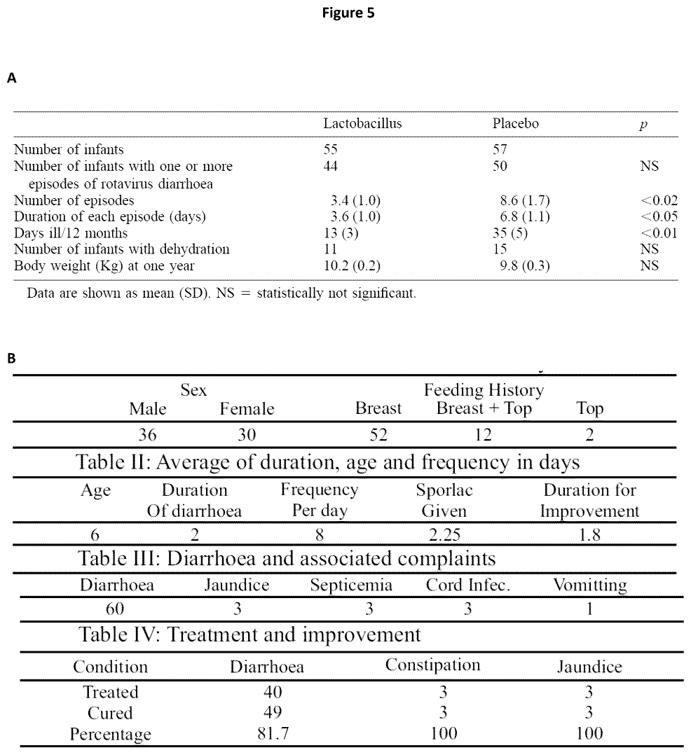

Symptomatic relief and classification according to Amsel's criteria after treatment on the basis of the treatment regimen and characteristics are shown in FIGS. 4A and B, respectively. The metronidazole+B. coagulans arm showed better success (86.6%) in treating and minimizing recurrence of vaginal infections and, consequently, symptoms associated with BV. See Rani et al., The Efficacy of Probiotic B. Coagulans (snz-1969) Tablets in the Treatment of Recurrent Bacterial Vaginosis, International Journal of Probiotics and Prebiotics Vol. 12, No. 4, pp. 175-182, 2017, herein incorporated by reference in its entirety).

### Example 5: Effect of Bacillus coagulans (Lactobacillus sporogenes) in Neonatal and Infantile Diarrhea

One hundred and twelve newborn infants in rural India were randomized to receive a daily oral dose of 100 million Lactobacillus sporogenes (SNZ 1969) or a placebo for one year. Morbidity was monitored each week for 12 months. Ninety-four (84%) experienced diarrhea due to rotavirus infection. The group fed L. sporogenes had fewer episodes of diarrhea (3.4±1.0 L. sporogenes group vs 8.6±1.7 in the placebo group, p<0.02) and less number of days of illness (13±3 days L. sporogenes group vs 35±5 days in the placebo group, p<0.01). The episodes of diarrhea were also of shorter duration (3.6±1.0 days L. sporogenes group vs 6.8±1.1 days in the placebo group, p<0.05). The number of infants who presented with mild to moderate dehydration was 11 in the treated group and in the placebo group; the difference was statistically not significant (p<0.05). There was a trend for body weight at one year to be higher (10.2±0.2) in the treated group compared with infants who were controls (9.8±0.3) but the difference was statistically not significant (p<0.05). These observations suggest that the prophylactic feeding of Lactobacillus has a preventive effect on the incidence and duration of acute rotavirus diarrhea (FIG. 5A). See Chandra, Effect of Lactobacillus on the incidence and severity of acute rotavirus diarrhoea in infants. A prospective placebo-controlled double-blind study, Nutrition Research (2002) 65-69, herein incorporated by reference in its entirety.

Diarrhea in newborn babies is also common. After the start of feeding following birth, the frequency of stool increases. The consistency of stool also changes usually on fifth day—known as ‘transitional stool’. One of the factors for this increase in frequency and change in consistency of stool is that the bacterial flora is not well established.

Lactobacillus sporogenes/Bacillus Coagulans SNZ 1969 (in the form of Sporlac tablets) was evaluated for its efficacy in stopping neonatal diarrhea. Cases were selected at random from the maternity ward of the Sassoon General Hospital, Pune for a total of 66. The majority of cases (60) experienced neonatal diarrhea. Only babies having more than 6 watery stools per day were selected for the trial. These babies were thoroughly examined for cord infection, pyoderma, and lung infection. They were also examined for jaundice and associated complaints. The feeding history was also noted.

A dose of about 5 million B. coagulans spores per kg body weight was explored. As the weight of a newborn baby is around 2 to 3 kg, only a ¼ (quarter) tablet of Sporlac, was given once a day to each newborn (each Sporlac tablet contains approximately 60 million spores). The results (shown in FIG. 5B) indicate that the B. coagulans SNZ 1969 was safe and had a high success rate in treating neonatal diarrhea; the overall success rate was 81.7% experienced at least an improvement in diarrhea symptoms, and all newborns experiencing constipation and of jaundice were treated/cured (100%). The average duration for complete recovery was 1.8 days, and dehydration was significantly prevented. See Dhongade and Anjaneyulu, Sporlac in Neonatal Diarrhoea, Maharashtra Medical Journal, Vol. XXIII, No. 11, February 1977, herein incorporated by reference in its entirety.

### Example 6: Effect of Lactobacillus paracasei in Immune Function in Elderly Subjects

Forty-two participants in two nursing homes who were ≥65 years of age were randomized to receive a jelly containing 10 billion heat-killed Lactobacillus paracasei MCC1849 cells (LP group) or a placebo jelly without lactobacilli (placebo group) for 6 weeks. Three weeks after beginning jelly intake, all subjects received an influenza vaccination (A/H1N1, A/H2N3 and B). Blood samples were collected before and after the treatment period. There were no significant differences in immune parameters, including in antibody responses against the vaccination, between the groups. In the subgroup of the oldest old, defined as ≥85 years of age (n=27), the antibody responses to the A/H1N1 and B antigens were improved in the LP group in comparison to the placebo group; the placebo group actually demonstrated impaired antibody response. No significant effects of L. paracasei MCC1849 were observed in the other elderly. See Maruyama et al., The effects of non-viable Lactobacillus on immune function in the elderly: a randomised, double-blind, placebo-controlled study, Int J Food Sci Nutr. 67 (1): 67-73 (2016), herein incorporated by reference in its entirety.

A randomized open-label trial was conducted from March 2017 to October 2017 on 62 older Japanese adult participants (aged ≥65 years) who reported of daily fatigue. Participants were equally, but randomly, assigned to either the test or control beverage group (31 in each group) using a computerized random-number generator. They consumed one bottle of their assigned drink each day for 4 weeks. The effects on improving the immune system were assessed based on the immunological vigor scores, a questionnaire, salivary test, blood test, and physical examination at baseline and at 4 weeks. Results Sixty subjects were analyzed as per-protocol set (n=30 each). After consuming the drink, immune strength scores in the test beverage group were significantly higher than those in the control beverage group at 4 weeks (P<0.05). Consumption of an oral nutritional supplement drink containing Lactobacillus paracasei MCC1849 for 4 weeks possibly improved the immune system of elderly people by preventing the decline of immune strength and improving quality of life-related subjective symptoms of fatigue. See Shimono et al., Effects of an Oral Nutritional Supplement Drink Containing Lactobacillus paracasei MCC 1849 on Improving the Immune System of Elderly People—A Randomized Open label Trial, Jpn Pharmacol Ther. 47 (1): 97-(2019), herein incorporated by reference in its entirety.

### Example 7: Effect of Bacillus coagulans on Oral Health

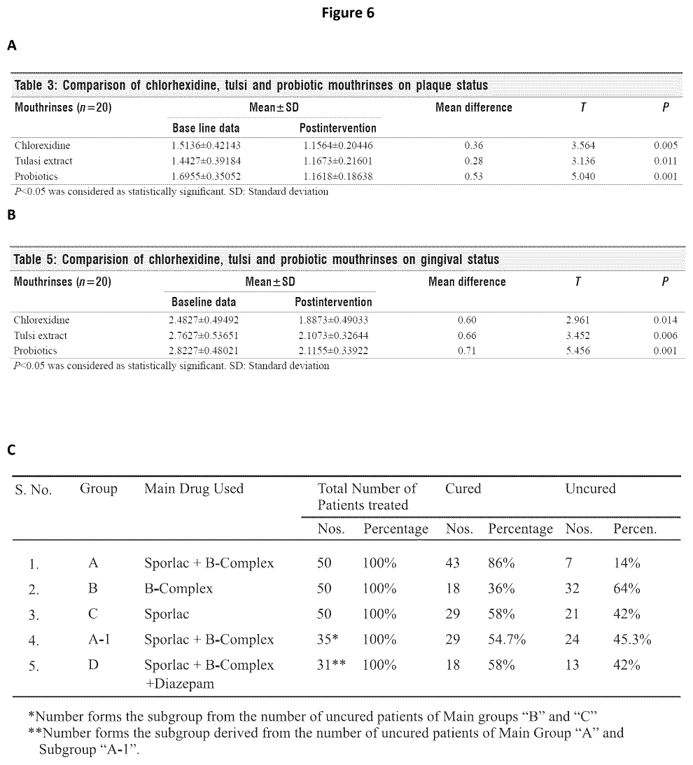

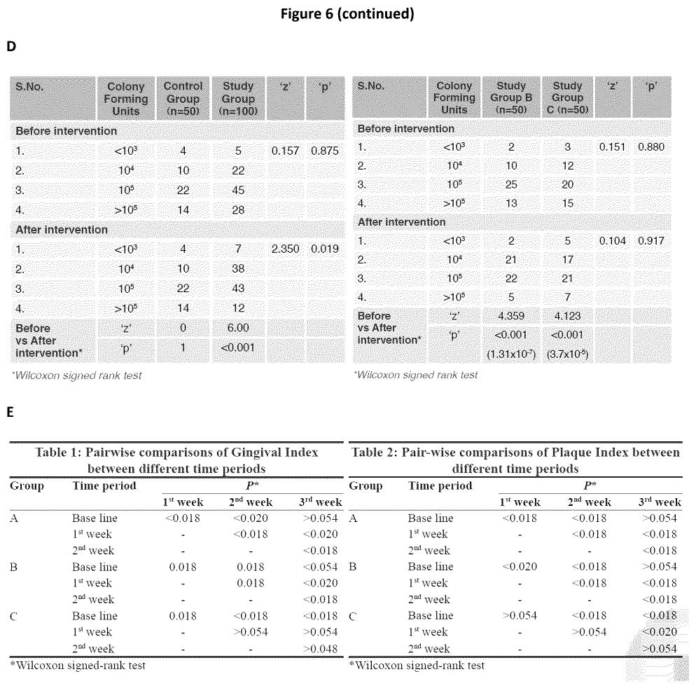

Probiotics and tulsi extract mouth rinses are a breakthrough approach to preventing plaque and gingivitis through their different defense mechanisms. Comparison of the effectiveness of chlorhexidine (CHX, 0.2%), tulsi extract (1 g Tulsi extract, 3 g Tween 80, 0.2 g menthol, 3 g sorbitol in 100 mL of water) and probiotic mouth rinses (1 g powder of 150 million spores of bacillus Coagulans in 20 mL of water) among 12-15-year-old school children of Mysore, India were studied and compared in a triple-blinded, randomized, and controlled clinical trial-concurrent parallel design. The study was done in a school which was selected by the lottery method. A total of 60 children who fulfilled the eligibility criteria were randomly selected for the study. After baseline examination, subjects were divided and assigned by computer generated table into three groups-Group A: 0.2% CHX mouth rinse, Group B: tulsi extract mouth rinse, and Group C: probiotic (B. coagulans) mouth rinse. There was a significant reduction in plaque and gingivitis among all three study products (P<0.05). Compared to baseline data, plaque and gingivitis reduction was more pronounced in probiotic mouth rinse compared to chlorhexidine and tulsi extract mouth rinses (FIG. 6A, 6B). All the study products, i.e., CHX, tulsi extract, and probiotic mouth rinses showed a significant reduction in plaque and gingivitis compared to baseline scores. See Manjunathappa et al., Effect of Tulsi Extract and Probiotic Mouthrinse on Plaque and Gingivitis among School Children—A Randomized Controlled Trial, Journal of Datta Meghe Institute of Medical Sciences University, Vol. 15, Issue 1, January-March (2020), herein incorporated by reference in its entirety.

Additionally, about one in five suffer from recurrent aphthous ulceration in their life, with little difference in incidence among the two sexes. No causative agent has been identified to date, but some risk factors have been suggested, such as vitamin B-Complex deficiency, emotional and environmental stress, food or drug allergy, endocrine imbalance, and blood dyscrasias. Lactobacillus sporogenes (B. coagulans) helps the restoration of vitamin B-Complex deficiency, and was selected for study as a potential treatment of aphthous ulcerations. A total of 150 patients suffering from recurrent aphthous ulcerations were included in the trials, irrespective of their age, sex, and race, from the outpatient sections of the Departments of Dental Surgery and Medicine of the Nehru Hospital associated with BRD Medical College Gorakhpur. Patients having underlying etiological factors other than vitamin B deficiency were excluded. Subjects were divided in 4 groups, and given supportive treatment (Diazepam 5 mg/tablet, twice a day for 5 days) if and when thought appropriate (sub group D):
Main group “A”: 50 patients treated with Sporlac (60 million spores of L. sporogenes/tablet, 2 tablets/day for 5 days)+B-complex vitamin therapy
Main group “B”: 50 patients B-complex vitamin therapy only
Main group “C”: 50 patients treated with Sporlac only
Sub group A-1:39 patients derived from failure of Main groups “B” and “C” that were later on treated as in Main group “A”
Sub group “D”: 31 patients derived from failure of main group “A” and sub-group of Main group “A-1” that were later on treated with diazepam, B-complex and Sporlac. Maximum cure rate was observed in Group A (86%); then in group C (58%), and the group B (18%).

Subgroup A-1 showed a cure rate of 54.7%, while subgroup D had a cure rate of 58% (FIG. 6C). See Sharma et al., Clinical trial of Sporlac in the treatment of recurrent aphthous ulceration, U.P. State Dental Journal Vol. 11, January (1980), 7-12, herein incorporated by reference in its entirety.

Another study was carried out to evaluate the effect of B. coagulans on salivary mutans streptococci counts in children and to explore its anti-caries potential. A placebo-controlled study was undertaken with 3 parallel arms comprising a total of 150 healthy children (7-14 years). The subjects were randomly divided in the groups (each comprising 50 children): group A: placebo powder; group B: a freeze-dried powdered combination of Lactobacillus rhamnosus and Bifidobacterium species (Darolac (Aristo Pharmaceuticals), 1 g powder of 1.25 billion freeze-dried bacterial combination); group C: a freeze-dried powdered preparation of Bacillus coagulans (Sporolac (Uni-Sankyo Ltd) 1 g powder with 150 million spores of B. coagulans). Subjects were instructed to mix the preparation in 20 ml of water and to follow a swish and swallow method for 14 days. Mutans streptococci colony counts per ml of saliva were performed on Mitis-Salivarius Bacitracin agar collected on the first day and 14 days post-intervention. A statistically significant reduction (p<0.001) in salivary mutans streptococci counts was recorded in both groups B and C after 14 days of probiotic ingestion. In conclusion, a cost-effective probiotic such as B. coagulans might be a subject for further research for prevention of caries in children (FIG. 6D). See Jindal et al., A comparative evaluation of probiotics on salivary mutans streptococci counts in Indian children, European Archives of Paediatric Dentistry, 12 (4) (2011), herein incorporated by reference in its entirety).

Another study aimed to evaluate the effectiveness of freeze-dried powdered B. coagulans on gingival status and plaque inhibition among 12-15-year-old schoolchildren. This randomized controlled trial was conducted among 12-15-year-old schoolchildren in Jaipur. Commercially available freeze-dried probiotics containing Lactobacillus acidophilus, Bifidobacterium longum, Bifidobacterium bifidum and Bifidobacterium lactis (Prowel, Alkem Laboratories), lactic acid bacillus only (Sporolac, Sangyo), and a placebo powder calcium carbonate 250 g (Calcium Sandoz, Novartis) were assigned to two intervention groups and a placebo group each comprising 11 school children. All subjects were instructed to mix the powder in 30 ml of water and swish once daily for 3 min, for 3 weeks. Periodontal clinical parameters were assessed by examining the subjects for Turesky-Gilmore-Glickman plaque index (PI) (Modification of Quigley-Hein PI) and gingival index at baseline, 7th day, 14th day, and 21st day. For both the probiotic groups, a statistically significant reduction (P<0.05) in gingival status and plaque inhibition was recorded up to 2nd week of probiotic ingestion. However, no significant difference was observed in the placebo group. The use of probiotic mouth rinses improves the oral health in children by significantly reducing the plaque and gingival scores (FIG. 6E). See Yousuf et al., Effect of Freeze-Dried Powdered Probiotics on Gingival Status and Plaque Inhibition: A Randomized, Double-blind, Parallel Study, Contemporary Clinical Dentistry Vol. 8 (1) (2017), herein incorporated by reference in its entirety.

### Example 8: Effect of Bacillus coagulans on Gastrointestinal Discomfort

Bacillus coagulans-based probiotics are believed to restore gut microbiota and alleviate symptoms of gastrointestinal (GI) discomfort. This study evaluated the efficacy and safety of SNZ 1969 in individuals with GI discomfort. This was a single-center, randomized, placebo-controlled, parallel-arm, double-blind study. Participants with GI discomfort (n=30 in each arm) without a specific pathology were randomized to receive B. coagulans-SNZ 1969, TriBac, or placebo, once daily after a major meal, for 30 days. Symptoms were assessed using the Severity of Dyspepsia Assessment (SODA) scale, Gastrointestinal Symptom Rating Scale (GSRS), and Short Form 36 (SF-36) at baseline, 9 day 15, day 30, and 7 days after the end of treatment. A total of 29 participants from SNZ 1969 and 28 from the placebo group completed the study.

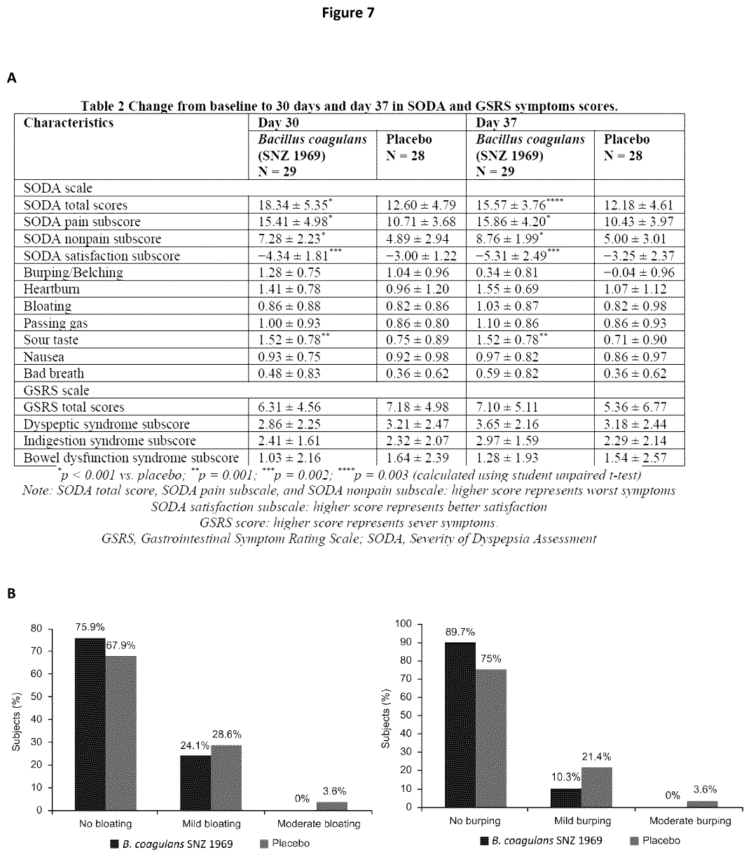

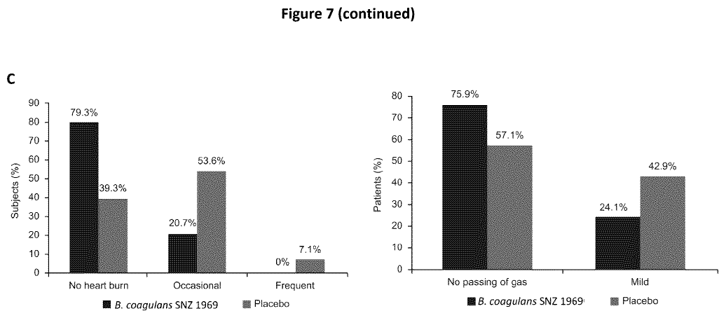

The reduction in total SODA score from baseline at day 30 was significantly higher for SNZ 1969 compared with placebo (18.34±5.35 vs. 12.60±4.79; p<0.001) (FIG. 7A). The mean baseline and day 30 SODA pain intensity subscores for SNZ 1969 were 29.34±1.57 and 13.93±4.42, respectively. The SODA pain intensity subscore reduction from baseline at day 30 was significantly higher for SNZ 1969 arm than in the placebo arm (15.41±4.98 vs. 10.71±3.68; p<0.001). The SODA nonpain symptom subscore reduction reflected relief from burping/belching, heartburn, bloating, passing of gas, sour taste, nausea, and bad breath. The reduction from baseline was significantly more in participants treated with SNZ 1969 compared with placebo at day 30 (7.28±2.23 vs. 4.89±2.94; p<0.001). Additionally, among the nonpain symptoms, a significantly greater reduction in sour taste was seen after treatment with SNZ 1969 than placebo 1.52±0.78 vs. 0.75±0.89; p=22 0.001). The SODA satisfaction subscore with abdominal discomfort significantly improved with SNZ 1969 treatment than placebo (−4.34±1.81 vs. −3.00±1.22; p=0.002). Significant improvement in SODA scale scores at day 30 compared with placebo was sustained after 7 days of discontinuing the SNZ 1969 treatment (p<0.05). These included total SODA score (15.57±3.76 vs. 12.18±4.61; p=0.003), SODA pain intensity subscore (15.86±4.20 vs. 10.43±3.97; p<0.001), SODA nonpain subscore (8.76±1.99 vs. 5.00±3.01; p<0.001), SODA satisfaction score (−5.31±2.49 vs. −3.25±2.37; p=0.002), and SODA sour taste (1.52±0.78 vs. 0.71±0.90; p<0.001). SNZ 1969 was found to be safe and effective in reducing GI discomfort, especially dyspepsia (FIG. 7). See Soman et al., Efficacy and Safety of Probiotic Bacillus coagulans-SNZ 1969 in Gastrointestinal Discomfort: A Randomized, Placebo-Controlled Study, International Journal of Health Sciences and Research Vol. 12; Issue: 3; March 2022, herein incorporated by reference in its entirety.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. An orally ingestible composition comprising:
an amount of gold kiwifruit powder,
an amount of Bacillus coagulans, and
an amount of heat-treated Lactobacillus paracasei.

2. The composition according to claim 1, wherein the gold kiwifruit powder comprises Livaux®.

3. The composition according to claim 1, wherein the Bacillus coagulans comprises SNZ 1969®.

4. The composition according to claim 1, wherein the Lactobacillus paracasei comprises MCC1849.

5. The composition according to claim 1, wherein the ratio of the amount of gold kiwifruit powder:the amount of Bacillus coagulans is between 15:1 and 35:1.

6. The composition according to claim 1, wherein the ratio of the amount of gold kiwifruit powder:the amount of Lactobacillus paracasei is between 5:1 and 20:1.

7. The composition according to claim 1, wherein the ratio of the amount of Lactobacillus paracasei:the amount of Bacillus coagulans is between 1.5:1 and 3:1.

8. The composition according to claim 1, wherein the orally ingestible composition is in the form of an individual oral dosage form.

9. The composition according to claim 1, wherein the individual oral dosage form is a gummy.

10. The composition according to claim 8, wherein the oral dosage form comprises between 75 mg and 700 mg gold kiwifruit powder; between 5 mg and 80 mg heat-treated Lactobacillus paracasei; and between 1 mg and 40 mg Bacillus coagulans.

11. The composition according to claim 8, wherein the oral dosage form comprises between about 100 million CFUs and 4 billion CFUs Bacillus coagulans.

12. The composition according to claim 10, wherein the oral dosage form comprises about 125 mg gold kiwifruit powder; about 10 mg Lactobacillus paracasei; and about 5 mg Bacillus coagulans.

13. The composition according to claim 11, wherein the oral dosage form comprises about 500 million CFUs Bacillus coagulans.

14. A method of increasing the amount of Faecalibacterium prausnitzii within a digestive tract of a human subject, wherein the method comprises orally administering, to the human subject, an orally ingestible composition according to claim 1.

15. A method of reducing constipation or relieving symptoms of constipation in a human subject, wherein the method comprises orally administering, to the human subject, an orally ingestible composition according to claim 1.

16.-17. (canceled)

18. A method of improving immune health of a human subject, wherein the method comprises orally administering, to the human subject, an orally ingestible composition according to claim 1.

19.-24. (canceled)

25. A method of improving oral health of a human subject, wherein the method comprises orally administering, to the human subject, an orally ingestible composition according to claim 1.

26.-29. (canceled)

30. A method of reducing bacterial vaginosis symptoms in a human female subject, wherein the method comprises orally administering, to the human female subject, an orally ingestible composition according to claim 1.

31. (canceled)

32. A method of reducing gastrointestinal discomfort in a human subject, wherein the method comprises orally administering, to the human subject, an orally ingestible composition according to claim 1.

33.-35. (canceled)
